(19) United States
(12) Patent Application Publication
(10) Pub. No.: US 2022/0338804 A1
(43) Pub. Date: Oct. 27, 2022

(54) OBJECTIVE ASSESSMENT OF PATIENT RESPONSE FOR CALIBRATION OF THERAPEUTIC INTERVENTIONS

(71) Applicants: Arizona Board of Regents on behalf of Arizona State University, Scottsdale, AZ (US); Aural Analytics, Inc., Scottsdale, AZ (US); Tamarisc Ventures LLC, Irvine, CA (US)

(72) Inventors: Visar BERISHA, Tempe, AZ (US); Julie LISS, Scottsdale, AZ (US); Daniel JONES, Phoenix, AZ (US); Seng TOH, Riverside, CA (US); David BATES, Brookline, MA (US)

(21) Appl. No.: 17/764,016

(22) PCT Filed: Sep. 28, 2020
(86) PCT No.: PCT/US2020/053059

§ 371 Date: Mar. 25, 2022

Related U.S. Application Data
(60) Provisional application No. 62/906,939, filed on Sep. 27, 2019.

(51) Int. Cl. A61B 5/00; G10L 25/66; G10L 15/22; G16H 20/10; G16H 50/30

(52) U.S. Cl. CPC A61B 5/4839; A61B 5/4803; A61B 5/4848; A61B 5/4824; G10L 25/66; G10L 15/22; G16H 20/10; G16H 50/30

(57) ABSTRACT

Systems and methods for objective assessment of patient response for calibration of therapeutic interventions are pro-vided. Analysis of a human patient's speech provides an objective measurement for pain or discomfort experienced by a patient. This speech analysis can then be used to provide personalized therapeutic interventions which more effectively address the needs of the patient. The speech analysis provides not only a personalized initial intervention, but in the case of ongoing interventions the speech analysis can further refine the intervention as a patient's response changes over time.

### RELATED APPLICATIONS

This application claims the benefit of provisional patent application Ser. No. 62/906,939, filed Sep. 27, 2019, the disclosure of which is hereby incorporated herein by reference in its entirety.

### FIELD OF THE DISCLOSURE

The present disclosure relates to measuring responses of human subjects to medical treatment.

### BACKGROUND

In a variety of medical and therapeutic interventions, objectively assessing pain and discomfort levels of patients would facilitate improved health outcomes. For example, in the administration of pain-relieving drugs it is desirable to determine an amount of a given drug necessary to relieve pain so that a precise amount of the drug can be administered and not more. Patient-controlled analgesia (PCA) devices allow a patient to administer a pain reliever (e.g., an opioid or other pain-relieving drug) via a programmable infusion pump. The traditional approach to operating a PCA device relies completely on the patient's assessment of their own pain levels. There are two prerequisites for effective opioid analgesia in this context: 1) the dosage must be titrated precisely to achieve an individualized pain relief response at the minimum effective analgesic concentration, and 2) the plasma opioid concentrations must remain constant to avoid peaks and troughs.

During programming, a PCA device has four variables that must be set: an initial loading dose, a demand dose, a lockout interval, a background infusion rate, and administration time limits (typically a 1-hour limit and a 4-hour limit). Traditionally, the initial loading dose is manually set by a nurse or doctor. The demand dose is an amount of pain reliever given to a patient when they activate a demand button in the pump. The background rate is a constant rate of analgesia administered to the patient, regardless of whether they press the button or not. The administration time limits impose constraints on how many times the patient can press the demand button. This programming design is constructed to achieve the goals of maintaining a stable minimum effective analgesic concentration with maximum pain relief, and prevent over-administration which could lead to potentially toxic drug plasma concentrations.

While the premise of the PCA device is that patients are empowered to self-administer analgesia until pain relief is achieved, the clinical reality is that most patients have a tendency to adhere to their own inherent maximum frequency of demands. This runs a risk of patient frustration when pain relief is not attained or maintained using the PCA device. Thus, the goal of achieving optimal effective analgesic plasma concentrations is critically dependent on understanding how that patient's pain level is related to the drug concentration.

FIG. 1 is a graphical representation of a theoretical intervention-response curve for patient pain response as a function of analgesic drug concentration. Previous studies have explored this relationship with the aim of generating such a theoretical curve. The intervention-response curve shows the minimum level of opioid concentration required to completely reduce a patient's pain. Table 1 below illustrates common concentrations which have been previously determined for opioid-naive patients across large populations:

TABLE 1
Common IV-Patient Controlled Analgesia
Regimens for Opioid-Naïve Patients

| | Demand | | Lockout | Continuous | |
|---|---|---|---|---|---|
| Opioid | dose | | (min) | basal* | |
| Morphine | 1-2 | mg | 6-10 | 0-2 | mg/h |
| Hydromorphone | 0.2-0.4 | mg | 6-10 | 0-0.4 | mg/h |
| Fentanyl | 20-50 | μg | 5-10 | 0-60 | μg/h |
| Sufentanil | 4-6 | μg | 5-10 | 0-8 | μg/h |
| Meperidine† | 10-20 | mg | 6-10 | 0-20 | mg/h |
| Tramadol | 10-20 | mg | 6-10 | 0-20 | mg/h |

*Continous basal infusions are not recommended for initial programming; †Meperidine should only be used in patients intolerant to all other opioids.

The traditional approach for programming and setting the PCA device relies on normative data from tables like Table 1 above. Furthermore, any customization of the PCA device parameters relies on a subjective assessment of pain.

### SUMMARY

Systems and methods for objective assessment of patient response for calibration of therapeutic interventions are provided. Analysis of a human patient's speech provides an objective measurement for pain or discomfort experienced by a patient. This speech analysis can then be used to provide personalized therapeutic interventions which more effectively address the needs of the patient. The speech analysis provides not only a personalized initial intervention, but in the case of ongoing interventions the speech analysis can further refine the intervention as a patient's response changes over time.

In this regard, embodiments disclosed herein elicit one or more initial speech samples before applying a therapeutic intervention, such as administration of a pain reliever (e.g., an analgesic drug). The therapeutic intervention is applied (e.g., an initial dose of the pain reliever), and one or more response speech samples are elicited. The initial speech sample(s) and the response speech sample(s) are analyzed to produce an intervention-response relationship (such as a curve, gradient, mathematical function, model, etc.), which can be used to provide a calibrated therapeutic intervention (e.g., an initial calibration of a patient-controlled analgesia (PCA) device). Further examples can provide continuous monitoring of the patient's response to the calibrated therapeutic intervention to further calibrate and personalize the intervention.

An exemplary embodiment provides a method for assessing a response of a human patient to a therapeutic intervention. The method includes receiving an initial speech sample of the human patient and administering the therapeutic intervention to the human patient. The method further includes, after administering the therapeutic intervention, receiving a first response speech sample of the human patient. The method further includes analyzing the initial speech sample and the first response speech sample to produce an intervention-response relationship for the human patient.

Another exemplary embodiment provides a method for calibrating an automated therapeutic device. The method includes receiving an initial speech sample and receiving a first response speech sample after administering a therapeutic intervention. The method further includes analyzing the initial speech sample and the first response speech sample to produce an intervention-response relationship. The method further includes calibrating the automated therapeutic device according to the intervention-response relationship.

Another exemplary embodiment provides a system for administering a therapeutic intervention to a human patient. The system includes an intervention administering device and a processing device. The processing device is configured to receive an initial speech sample of the human patient and cause the intervention administering device to administer the therapeutic intervention to the human patient. The processing device is further configured to receive a first response speech sample of the human patient after administering the therapeutic intervention and analyze the initial speech sample and the first response speech sample to produce an intervention-response relationship for the human patient.

In another aspect, any one or more aspects or features described herein may be combined with any one or more other aspects or features for additional advantage.

Other aspects and embodiments will be apparent from the detailed description and accompanying drawings.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a graphical representation of a theoretical intervention-response curve for patient pain response as a function of analgesic drug concentration.

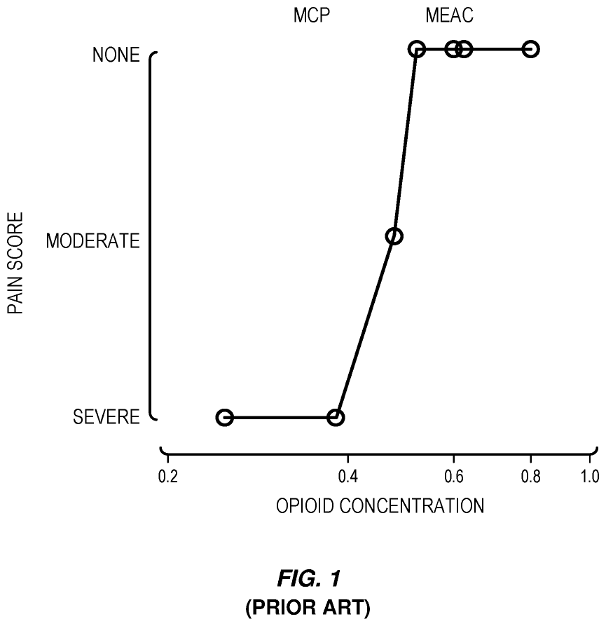

FIG. 2 is a schematic diagram of an exemplary objective approach for assessing a response of a patient to a therapeutic intervention.

FIG. 3 is a schematic diagram of an application of the objective approach of FIG. 2 to calibrating an intervention administering device, such as a patient-controlled analgesia (PCA) device.

FIG. 4 is a schematic diagram of producing a speech-pain gradient for the PCA device of FIG. 3.

FIG. 5 is a schematic diagram of calibration of the PCA device of FIG. 3.

FIG. 6 is a schematic diagram of a generalized representation of an exemplary computer system that could be used to perform any of the methods or functions described herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Systems and methods for objective assessment of patient response for calibration of therapeutic interventions are provided. Analysis of a human patient's speech provides an objective measurement for pain or discomfort experienced by a patient. This speech analysis can then be used to provide personalized therapeutic interventions which more effectively address the needs of the patient. The speech analysis provides not only a personalized initial intervention, but in the case of ongoing interventions the speech analysis can further refine the intervention as a patient's response changes over time.

In this regard, embodiments disclosed herein elicit one or more initial speech samples before applying a therapeutic intervention, such as administration of a pain reliever (e.g., an analgesic drug). The therapeutic intervention is applied (e.g., an initial dose of the pain reliever), and one or more response speech samples are elicited. The initial speech sample(s) and the response speech sample(s) are analyzed to produce an intervention-response relationship (such as a curve, gradient, mathematical function, model, etc.), which can be used to provide a calibrated therapeutic intervention (e.g., an initial calibration of a patient-controlled analgesia (PCA) device). Further examples can provide continuous monitoring of the patient's response to the calibrated therapeutic intervention to further calibrate and personalize the intervention.

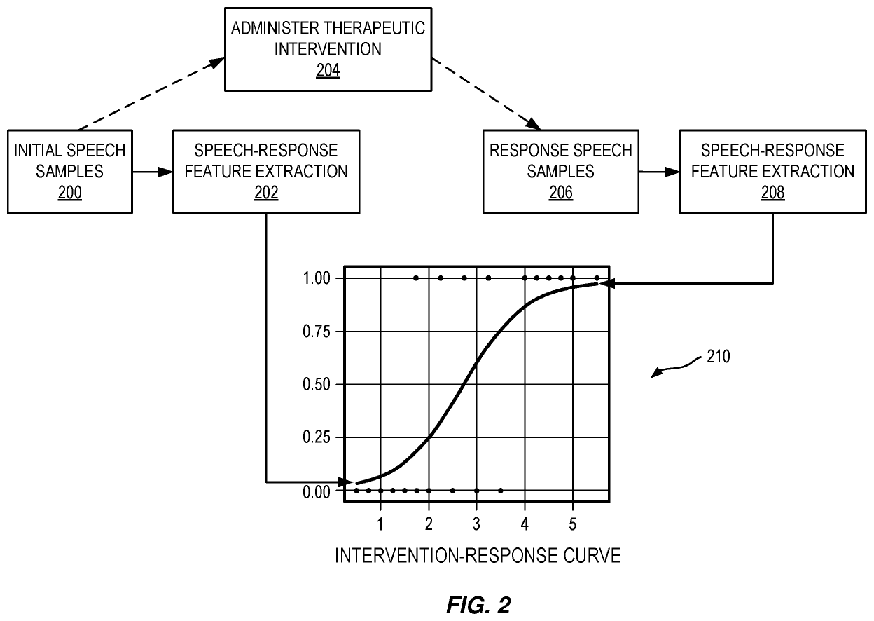

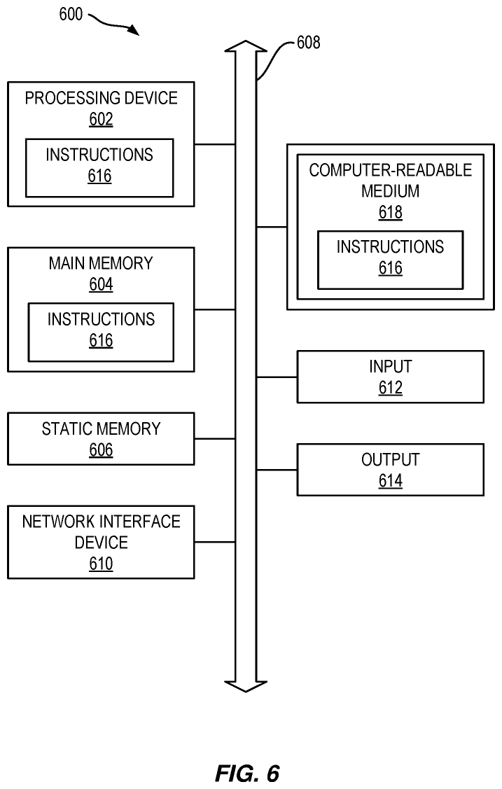

FIG. 2 is a schematic diagram of an exemplary objective approach for assessing a response of a patient to a therapeutic intervention. The proposed objective approach begins with receiving one or more initial speech samples of a human patient (block 200). In some examples, the one of more initial speech samples are elicited from the patient according to a model speech sample. For example, an output device (e.g., a display device or audio device) may provide a transcript for the patient to read to facilitate analysis of the elicited speech. In some examples, spontaneous speech samples are received. In some embodiments, the one or more initial speech samples are stored in a memory (e.g., a memory of a computing device such as described below with respect to FIG. 6). The initial speech samples are analyzed (e.g., by a processing device), and speech-response features are extracted from the initial speech samples (block 202). In some embodiments, the speech response features are stored in the memory in addition to or as an alternative to storing the initial speech samples.

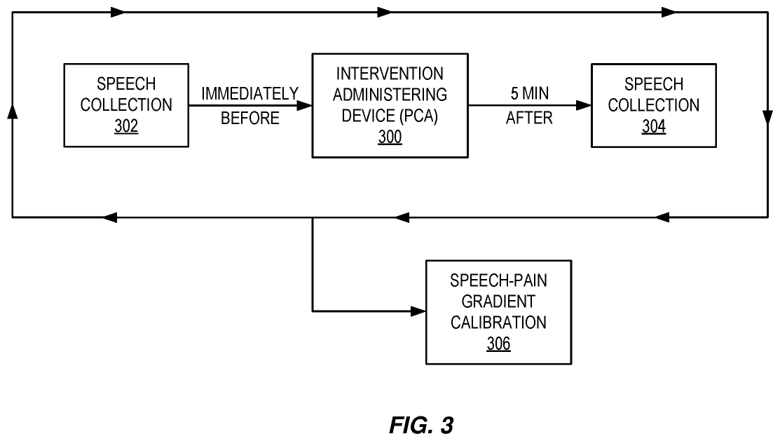

After the initial speech samples are received, a therapeutic intervention is administered to the patient (block 204). The therapeutic intervention can be administration of a drug (e.g., using a PCA device as further described below with respect to FIGS. 3-5), physical therapy, speech therapy, mental health intervention, surgical intervention, or other therapeutic interventions. After a given amount of time, one or more response speech samples are received (e.g., in a manner similar to the initial speech samples) (block 206). The given amount of time may be an appropriate time for the therapeutic intervention to take effect. In some cases, multiple response speech samples are taken at intervals to provide additional data points during the therapeutic intervention. The response speech samples are analyzed (e.g., by a processing device), and speech-response features are extracted from the response speech samples (block 208). In some embodiments, the response speech samples and/or the extracted speech response features are stored in the memory.

With initial speech samples and response speech samples received, the speech response features which have been extracted are further analyzed (e.g., by a processing device) to produce an intervention-response relationship 210 (such as a curve, gradient, mathematical function, model, etc.) for the patient. This provides a logistical model for further therapeutic intervention based on the patient's response. The logistic model could be anything from a simple linear model to a more complex pre-trained neural network (e.g., a deep neural network (DNN)). In some examples, a DNN could be trained using data from a large number of patients, but is here calibrated and adapted with intervention-response data from this particular patient.

The intervention-response relationship 210 can then be used to calibrate and personalize further therapeutic interventions according to the intervention-response relationship 210. In further examples, the patient's response to the therapeutic intervention can be monitored by receiving and analyzing additional response speech samples (e.g., during and after subsequent administration of the therapeutic intervention). The calibration of the therapeutic intervention can be further refined based on these additional response speech samples.

The proposed objective approach for assessing patient response to therapeutic intervention can be applied to devices which automatically administer the therapeutic intervention, as well as in concert with input and interventions provided by medical professionals. For example, this approach can provide an initial recommendation for the intervention, which may be subject to further input by such medical professionals. In addition, this approach can be used to facilitate improved outcomes where such medical professionals provide the intervention.

In some embodiments, the intervention-response relationship 210 is produced by analyzing additional objective data in conjunction with the initial speech sample(s) (block 200) and the response speech sample(s) (block 206). For example, embodiments may receive one or more of a video sample of the human patient, a facial scan of the human patient, eye movement of the human patient, a thermal sample of the human patient, a writing sample of the human patient, a heart rate of the human patient, or respiration data of the human patient. These may provide additional objective data points for analyzing the patient's response to pain and produce a more accurate intervention-response relationship 210.

FIG. 3 is a schematic diagram of an application of the objective approach of FIG. 2 to calibrating an intervention administering device 300, such as a PCA device. The intervention administering device 300 can assist in providing pain relief or another desired patient response. For example, a PCA device allows a patient to administer a pain reliever (e.g., an opioid or other pain-relieving drug) via a programmable infusion pump. Traditionally, the pump is programmed via normative data for different patient populations or through subjective assessment of the patient's pain scores. Embodiments disclosed herein can modify or augment these existing systems by providing an objective assessment of the patient's pain levels through speech analysis. These objective measures of pain can be used to program the PCA device in a personalized way.

In this regard, a patient can provide an initial speech sample to the intervention administering device 300 (block 302). The intervention administering device 300 can provide an initial therapeutic intervention (e.g., administration of a pain reliever or other drug, physical therapy, speech therapy, mental health intervention, surgical intervention, etc.). After the initial therapeutic intervention is provided, the patient provides one or more response speech samples, which can indicate how the patient has responded to the therapeutic intervention (also referred to as a response speech sample) (block 304). In some embodiments, the initial speech sample and the response speech sample are stored in a memory. The initial speech sample and the response speech sample(s) are analyzed (e.g., by a processing device) to produce an intervention-response relationship, which can be used to calibrate the intervention administering device 300 (block 306).

For example, a PCA device can be initially programmed using a traditional approach (e.g., with dose variables based on normative data). The patient is connected to the PCA device. The patient provides a short speech sample (e.g., 30-60 seconds) immediately before pressing a demand dose button on the PCA device. After a period of time (e.g., after a predicted time for the drug to take effect or when the patient feels that the pain has subsided), the patient provides another short speech sample. This process can then continue several times until there is sufficient data available to generate a speech-pain gradient. This is evaluated by a speech-pain gradient calibration system that processes the speech samples in the background. The speech-pain gradient is then used to adapt dosing variables for the drug for the remainder of the session.

In some embodiments, the calibration process of FIG. 3 is performed repeatedly during administration of the therapeutic intervention (e.g., periodically or triggered by an event). In some instances, a new initial speech sample is collected (block 302) before another intervention (e.g., before a demand dose of a drug), and a new response speech sample is collected (block 304), with both speech samples being used to recalibrate the intervention administering device 300 (block 306). In other instances, block 302 is omitted and response speech samples are collected periodically or in response to a triggering event (block 304) and used to recalibrate the intervention administering device 300 (block 306).

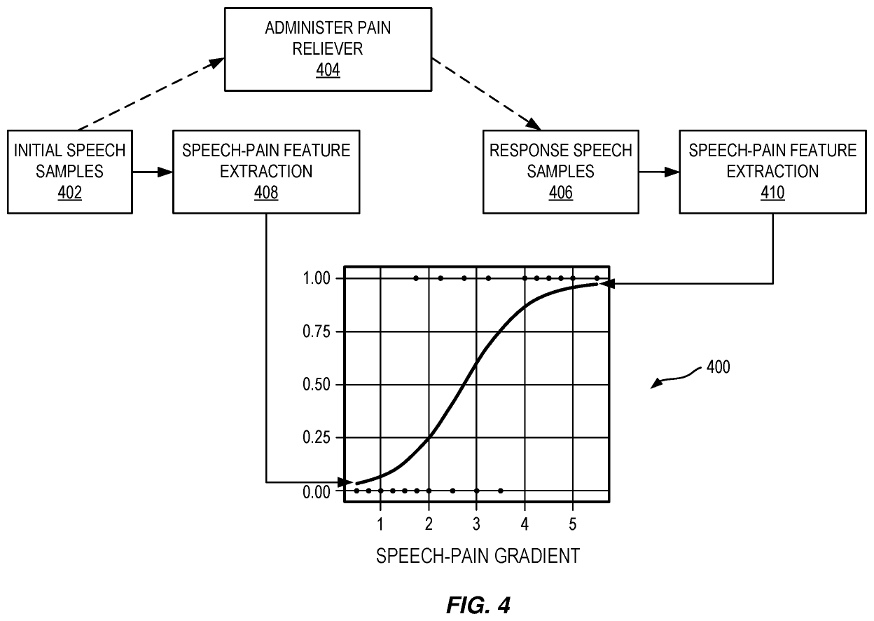

FIG. 4 is a schematic diagram of producing a speech-pain gradient 400 for a PCA device. The speech-pain gradient 400 is produced with an algorithm which operates on initial speech samples (block 402) from a PAIN condition (e.g., pre administration of a pain reliever or other drug by the PCA device (block 404)) and response speech samples from a NO-PAIN condition (post administration of the drug by the PCA device (block 404)) as new response speech samples (block 406) are collected. A set of relevant speech features are extracted from the speech samples from both groups (blocks 408 and 410).

These features are the input to an optimization algorithm that aims to learn a within-subject speech-pain gradient 400 using a mathematical function. This function can be a simple logistic sigmoid or can be a pre-trained DNN or other neural network (e.g., pre-trained based on data from a large set of speech-pain scores collected over time from other patients). In some examples, a personalized speech-pain gradient 400 is produced without reference to previous data. In some cases, a model that has been pre-trained on a large corpus of speech-pain scores is adapted to the patient based on the initial speech samples and the response speech samples.

In some cases, this algorithm is applied to several rounds of data collection, with two possible outcomes. First, the algorithm may automatically determine that speech is not a reliable way to assess pain for this patient and the process stops. Second, the algorithm may determine that speech is a reliable predictor of pain for this patient. If speech is a reliable predictor of pain, the algorithm can continue to calibrate the PCA device based on the speech-pain gradient.

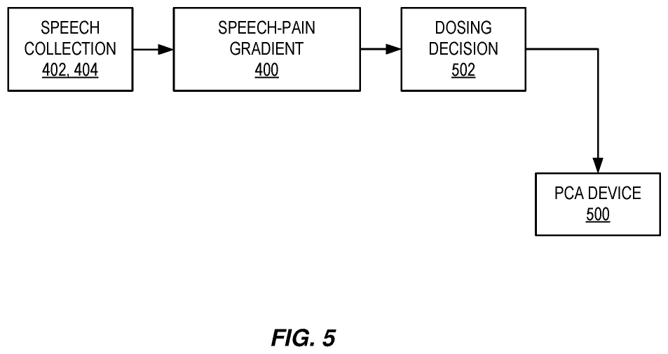

FIG. 5 is a schematic diagram of calibration of the PCA device 500 according to the teachings of FIG. 4. The speech-pain gradient 400 described above can be integrated into operation of the PCA device 500. In a traditional PCA device, after the initial setting of the PCA device using traditional methods, a medical professional (e.g., a nurses or doctor) has the option of reprogramming the dosing levels depending on the patient response to the drug (e.g., lower the dose if the patient is sedated, increase the dose if the patient is feeling pain).

The objective speech-pain gradient 400 provides objective criteria for calibration of the PCA device 500 from the speech samples collected at blocks 402 and 406. Calibration can be done automatically through the development of algorithms that optimally map perceived pain levels to dosing levels, or it can be done by a medical professional provided with the output of the speech-pain gradient 400 and changing the dosing levels accordingly (block 502). Thus, one or more of the following dosing variables can be modified using this approach: demand dose, lockout interval, background infusion rate, and one or more administration time limits (e.g., a 1-hour time limit and a 4-hour time limit). In addition, an initial loading dose for a subsequent administration of the PCA device 500 for the patient can also be modified using this approach.

FIG. 6 is a schematic diagram of a generalized representation of an exemplary computer system 600 that could be used to perform any of the methods or functions described above, such as assessing a response of a human patient to a therapeutic intervention or calibrating an automated therapeutic device. In some examples, the intervention administering device 300 of FIG. 3 (e.g., the PCA device 500 of FIG. 5) is implemented as the computer system 600. In some examples, the intervention administering device 300 is coupled to the computer system 600. In this regard, the computer system 600 may be a circuit or circuits included in an electronic board card, such as, a printed circuit board (PCB), a server, a personal computer, a desktop computer, a laptop computer, an array of computers, a personal digital assistant (PDA), a computing pad, a mobile device, or any other device, and may represent, for example, a server or a user's computer.

The exemplary computer system 600 in this embodiment includes a processing device 602 or processor, a main memory 604 (e.g., read-only memory (ROM), flash memory, dynamic random access memory (DRAM), such as synchronous DRAM (SDRAM), etc.), and a static memory 606 (e.g., flash memory, static random access memory (SRAM), etc.), which may communicate with each other via a data bus 608. Alternatively, the processing device 602 may be connected to the main memory 604 and/or static memory 606 directly or via some other connectivity means. In an exemplary aspect, the processing device 602 could be used to perform any of the methods or functions described above.

The processing device 602 represents one or more general-purpose processing devices, such as a microprocessor, central processing unit (CPU), or the like. More particularly, the processing device 602 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 602 is configured to execute processing logic in instructions for performing the operations and steps discussed herein.

The various illustrative logical blocks, modules, and circuits described in connection with the embodiments disclosed herein may be implemented or performed with the processing device 602, which may be a microprocessor, field programmable gate array (FPGA), a digital signal processor (DSP), an application-specific integrated circuit (ASIC), or other programmable logic device, a discrete gate or transistor logic, discrete hardware components, or any combination thereof designed to perform the functions described herein. Furthermore, the processing device 602 may be a microprocessor, or may be any conventional processor, controller, microcontroller, or state machine. The processing device 602 may also be implemented as a combination of computing devices (e.g., a combination of a DSP and a microprocessor, a plurality of microprocessors, one or more microprocessors in conjunction with a DSP core, or any other such configuration).

The computer system 600 may further include a network interface device 610. The computer system 600 also may or may not include an input 612, configured to receive input and selections to be communicated to the computer system 600 when executing instructions. In some examples, the input 612 includes or is connected to an audio input device (e.g., a microphone) for receiving speech samples. The computer system 600 also may or may not include an output 614, including but not limited to a display, a video display unit (e.g., a liquid crystal display (LCD) or a cathode ray tube (CRT)), an alphanumeric input device (e.g., a keyboard), and/or a cursor control device (e.g., a mouse). For example, the output 614 may include or be connected to a display, speaker, or other output device which requests the human patient to elicit the speech sample(s), and may additionally provide a transcript or other model speech sample for the speech sample(s).

The computer system 600 may or may not include a data storage device that includes instructions 616 stored in a computer-readable medium 618. The instructions 616 may also reside, completely or at least partially, within the main memory 604 and/or within the processing device 602 during execution thereof by the computer system 600, the main memory 604, and the processing device 602 also constituting computer-readable medium. The instructions 616 may further be transmitted or received via the network interface device 610.

While the computer-readable medium 618 is shown in an exemplary embodiment to be a single medium, the term “computer-readable medium” should be taken to include a single medium or multiple media (e.g., a centralized or distributed database, and/or associated caches and servers) that store the one or more sets of instructions 616. The term “computer-readable medium” shall also be taken to include any medium that is capable of storing, encoding, or carrying a set of instructions for execution by the processing device 602 and that causes the processing device 602 to perform any one or more of the methodologies of the embodiments disclosed herein. The term “computer-readable medium” shall accordingly be taken to include, but not be limited to, solid-state memories, optical medium, and magnetic medium.

The operational steps described in any of the exemplary embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the exemplary embodiments may be combined.

Those skilled in the art will recognize improvements and modifications to the preferred embodiments of the present disclosure. All such improvements and modifications are considered within the scope of the concepts disclosed herein and the claims that follow.

What is claimed is:

1. A method for assessing a response of a human patient to a therapeutic intervention, the method comprising:
receiving (200) and storing in a memory (604, 606) an initial speech sample of the human patient;
administering (204) the therapeutic intervention to the human patient;
after administering the therapeutic intervention, receiving (206) and storing in the memory a first response speech sample of the human patient; and
analyzing, with a processing device (602) the initial speech sample and the first response speech sample to produce an intervention-response relationship (210) for the human patient.

2. The method of claim 1, further comprising providing (306) a calibrated therapeutic intervention according to the intervention-response relationship.

3. The method of claim 2, further comprising:
monitoring a response of the human patient to the calibrated therapeutic intervention by receiving (304) and analyzing one or more additional response speech samples; and
recalibrating (306) the calibrated therapeutic intervention responsive to the analyzing the one or more additional response speech samples.

4. The method of any one of claim 2 or 3, wherein the therapeutic intervention comprises administering (404) a drug to the human patient.

5. The method of claim 4, wherein providing the calibrated therapeutic intervention comprises setting at least one of an initial loading dose, a demand dose, a lockout interval, an infusion rate, or an administration time limit for the drug.

6. The method of any one of claim 4 or 5, wherein the drug is a pain reliever.

7. The method of claim 6, wherein analyzing the initial speech sample and the first response speech sample comprises analyzing a pain level of the human patient responsive to the administering (404) the pain reliever to the human patient.

8. The method of claim 7, wherein:
administering (204) the therapeutic intervention to the human patient comprises administering (404) a dose of the pain reliever according to normative data from other human patients; and
the method further comprises providing (306) a calibrated dose of the pain reliever according to the intervention-response relationship (210).

9. The method of any one of claims 6 to 8, wherein the pain reliever is administered (404) by a patient-controlled analgesia (PCA) device.

10. The method of any one of claims 6 to 8, wherein the pain reliever is administered (404) by a medical professional.

11. The method of any one of claims 1 to 10, further comprising receiving additional objective data for the human patient;
wherein the intervention-response relationship (210) is further produced by analyzing the additional objective data.

12. The method of claim 11, wherein the additional objective data comprises at least one of:
a video sample of the human patient;
a facial scan of the human patient;
eye movement of the human patient;
a thermal sample of the human patient;
a writing sample of the human patient;
a heart rate of the human patient; or
respiration data of the human patient.

13. A method for calibrating an automated therapeutic device (300), the method comprising:
receiving (302) and storing in a memory (604, 606) an initial speech sample from a human patient;
receiving (304) and storing in the memory a first response speech sample after administering a therapeutic intervention;
analyzing, with a processing device, the initial speech sample and the first response speech sample to produce an intervention-response relationship (210); and
calibrating (306) the automated therapeutic device according to the intervention-response relationship.

14. The method of claim 13, wherein:
the therapeutic intervention comprises administering a drug to the human patient; and
calibrating the automated therapeutic device comprises setting at least one of an initial loading dose, a demand dose, a lockout interval, an infusion rate, or an administration time limit for the drug.

15. The method of claim 13, wherein
the automated therapeutic device (300) comprises a patient-controlled analgesia (PCA) device (500); and
analyzing the initial speech sample and the first response speech sample comprises analyzing a pain level of the human patient in response to the PCA device administering a pain reliever.

16. The method of claim 13, further comprising:
after calibrating the automated therapeutic device, receiving (304) a second response speech sample;
analyzing the second response speech sample to produce an updated intervention-response relationship (210); and
recalibrating (306) the automated therapeutic device according to the updated intervention-response relationship.

17. The method of claim 13, further comprising receiving additional objective data comprising at least one of a video sample, a facial scan, eye movement data, a thermal sample, a writing sample, heart rate data, or respiration data, wherein the intervention-response relationship (210) is further produced by analyzing the additional objective data.

18. A system for administering a therapeutic intervention to a human patient, the system comprising:
an intervention administering device (300); and
a processing device (602) configured to:
receive (200) an initial speech sample of the human patient;
cause (204) the intervention administering device to administer the therapeutic intervention to the human patient;
receive (206) a first response speech sample of the human patient after administering the therapeutic intervention; and
analyze the initial speech sample and the first response speech sample to produce an intervention-response relationship (210) for the human patient.

19. The system of claim 18, further comprising an audio input device (612) configured to receive (200, 206) the initial speech sample and the first response speech sample.

20. The system of claim 18, further comprising an output device (614) configured to request the human patient to elicit the initial speech sample and the first response speech sample.

21. The system of claim 20, wherein:
the output device provides a selected model speech sample to the human patient; and
the processing device is configured to analyze the initial speech sample and the first response speech sample based on the selected model speech sample.
